SEQUENCE LISTING (4 sequences)

SEQ ID NO: 1 - protein, 805 aa, Homo sapiens [sequence omitted]

SEQ ID NO: 2 - protein, 702 aa, Homo sapiens
EEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLE

SEQ ID NO: 3 - protein, 1279 aa, SARS coronovirus
MPMGSLQPLATLYLLGMLVASVLASGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYTGGTETSQVAPA

SEQ ID NO: 4 - protein, 657 aa, SARS coronovirus
TLYLLGMLVASVLASGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGA

(19) United States
(12) Patent Application Publication
(10) Pub. No.: US 2005/0282154 A1
(43) Pub. Date: Dec. 22, 2005

(54) Angiotensin-converting enzyme-2 as a receptor for the SARS coronavirus

(75) Inventors: Michael R. Farzan, Cambridge, MA (US); Wenhui Li, Boston, MA (US); Michael J. Moore, Cambridge, MA (US)

Correspondence Address: FITCH, EVEN, TABIN & FLANNERY, P. O. BOX 65973, WASHINGTON, DC, 20035, US

(73) Assignee: The Brigham and Women's Hospital, Inc.

(21) Appl. No.: 10/957,880

(22) Filed: Oct. 5, 2004

Related U.S. Application Data
(60) Provisional application No. 60/508,281, filed on Oct. 6, 2003.

(51) Int. Cl. C12Q 1/70; C12N 7/01; C12N 15/86

(52) U.S. Cl. 435/5; 435/235.1; 435/456

(57) ABSTRACT

The present invention is based upon the identification of human angiotensin-converting enzyme-2 (ACE-2) as a functional receptor for the SARS coronavirus. Transfection of cells with ACE-2 confers upon them the ability to support viral replication. In addition, assays performed using ACE-2 together with the S protein of the SARS virus or a fragment derived from the S protein can be used to identify inhibitors that block the interaction between virus and host cell.

### CROSS REFERENCE TO RELATED APPLICATIONS

The present application claims the benefit of U.S. provisional application No. 60/508,281 filed Oct. 6, 2003.

### FIELD OF THE INVENTION

The present invention is concerned with a host cell receptor recognized by the virus causing SARS. This receptor may be used to develop cell lines capable of maintaining viral growth and in the identification of therapeutic agents that block virus from entering cells.

### BACKGROUND OF THE INVENTION

Severe Acute Respiratory Syndrome (SARS) is a highly contagious and potentially lethal viral disease characterized by pronounced respiratory symptoms and pneumonia (Avendeno, et al., Can. Med. Assoc. J. 168:1649-1660 (2003)). The rapid emergence and spread of this disease has led to intense efforts to develop effective methods of treatment. These efforts have led to the isolation of a unique coronavirus that causes SARS and to the complete sequencing of this virus's genome (Drosten, et al., N. Eng. J. Med. 348:1967-1976 (2003); Marra, et al., Science 300:1399-1404 (2003); Rhota, et al., Science 300:1394-1399 (2003)).

One approach to the treatment SARS infections is to interfere with the process by which the virus enters host cells. This process is mediated by a surface protein, designated as “S,” which is analogous to the HIV-1 envelope glycoprotein (Gallagher, et al. Virology 279:371-374 (2001)). Some coronaviruses cleave the S protein into two fragments called S1, which mediates receptor binding, and S2, which mediates fusion of the virus to the host cell membrane (Sturman, et al., Adv. Exp. Med Biol. 173:25-35 (1984); Jackwood, et al., Avian Dis. 45:366-372 (2001)). In other coronaviruses, including the one causing SARS, the S protein is not cleaved (Spaan, et al., J. Gen. Virol. 69:2939-2952 (1988)). Nevertheless, the S1 and S2 domains can be identified and initial receptor binding is still mediated by S1 (Bonavia, et al., J. Virol. 77:2530-2538 (2003); Breslin, et al., J. Virol. 77:4435-4438 (2003); Kubo, et al., J. Virol. 68:5403-5410 (1994)).

Regardless of the form of S protein produced, coronaviruses fall into two main types with respect to cellular entry (Holmes, et al., Adv. Exp. Med. Biol. 342:261-266 (1993)). One type, represented by the mouse hepatitis virus, utilizes members of the immunoglobulin superfamily of receptors (Dveksler, et al., J. Virol. 67:1-8 (1993); Dveksler, et al., J. Virol. 65:6881-6891 (1991); Nedellec, et al., J. Virol. 68:4525-4537 (1994)). The second type, represented by the human coronavirus 229E, transmissible gastroenteritis virus and feline infectious peritonitis virus, requires the zinc metalloprotease aminopeptidase N (APN, CD13) for cellular entry (Delmas, et al., Nature 357:417-420 (1992); Tresnan, et al., Adv. Exp. Med. Biol. 440:69-75 (1998); Yeager, et al., Nature 357:420-422 (1992)). Determining whether the coronavirus causing SARS uses similar methods for entering cells and identifying the host cell receptor that binds the SARS S protein is of great importance in the development of vaccines and new therapeutic agents.

### SUMMARY OF THE INVENTION

The present invention is based upon the discovery that the metallopeptidase angiotensin-converting enzyme-2 (ACE-2) serves as a mammalian cell receptor for the human SARS coronavirus. This conclusion is based upon experiments suggesting that ACE-2 is expressed in receptor-bearing Vero E6 cells and that it associates specifically with the S1 domain of the SARS S protein. The transfection of cells with sequences expressing ACE-2 confers upon them the ability to support the replication of the SARS virus. The full length human ACE-2 sequence is shown as SEQ ID NO: 1. Residues 1-21 are the signal sequence and get cleaved off. Residues 724-805 are the transmembrane and cytoplasmic region. Soluble ACE-2 includes residues 22-723 and is provided in the sequence listing as SEQ ID NO:2; the SARS S protein is SEQ ID NO:3,; and the SARS S1 protein is SEQ ID NO:4.

In its first aspect, the invention is directed to a method of growing the SARS coronavirus in cells. This is accomplished by first engineering the cells to stably express the human ACE-2 protein (see SEQ ID NO: 1). Molecular biology provides a number of methods for engineering cells to increase protein production and any may be used in the present instance. However, the preferred method is to transfect the cells with a vector in which a promoter is operably linked to a nucleotide sequence coding for ACE-2. The term “operably linked” means that the promoter and coding sequence are joined together in a manner that allows them to carry out their normal functions, i.e., transcription of the coding sequence is under the control of the promoter and the transcript produced is correctly translated into the desired product. The next step in the process involves culturing the cells under conditions that provide for their maintenance or growth. Standard conditions for culturing a variety of cells are well known in the art and may be employed for the present invention. After the engineered cells have been cultured, they are infected with the SARS coronavirus. The presence of the human ACE-2 protein on the cell surface allows the virus to enter into the cells and undergo replication. Virus produced in this manner may then be harvested using standard methodology. In general, mammalian cells are preferred for this process, and human 293 T cells, CHO cells and Vero cells are particularly preferred. One advantage of the procedure is that it allows cells that have been approved by the FDA for the production of clinical products to be used for manufacturing weakened forms of the SARS virus that may be used in vaccines. The methodology may also be used by researchers studying the biological characteristics of SARS.

The identification of the SARS receptor allows one of ordinary skill in the art to utilize a wide variety of assay methods for: a) studying receptor/ligand interaction; b) identifying agents that block this interaction; and c) developing assays for detecting the presence of virus. These assays may take many different forms, all of which are encompassed by the present invention. In one preferred assay, a test compound is examined for its ability to block the binding of the SARS S or S1 protein to cells bearing the receptor. This assay is of particular utility in the identification of therapeutic agents that can be administered to SARS patients.

In the most preferred embodiment, cells expressing the human ACE-2 protein on their surface are incubated with detectably labeled S protein or a detectably fragment of the S protein that maintains its ability to specifically bind to human ACE-2. In preferred embodiments, the intact S protein shown in SEQ ID NO:3 or the S1 fragment of SEQ ID NO:4 are used. It is also possible to perform assays using the virus itself. The term “detectably labeled” means that proteins are attached to a molecule that can be either directly or indirectly assayed using standard laboratory techniques. For example, protein or peptides may be attached to a radioactive isotope such as ¹²⁵I or to a fluorescent tag such as fluoresceineisothiocyanate (FITC). Peptides or proteins may also be detectably labeled by expressing them as part of a fusion protein in which they are attached to a marker amino acid sequence. A “marker amino acid sequence” is one that binds with high affinity to an antibody or other compound, thereby permitting detection. For example, peptides may be fused to the Fc fragment of IgG and the fusion peptide subsequently detected using a labeled antibody that binds with high affinity to Fc. The incubations will also include test compound. Preferably, the test compound will be examined at several different concentrations.

After incubation, unbound ligand is removed from cells and the amount of label bound is determined. A comparison is then made between these results and results obtained from cells incubated under similar conditions, but in the absence of test compound. If the amount of binding observed in the presence of the test compound is lower than in the absence of test compound, this is an indication that the compound blocks the binding of the SARS S protein to the human ACE-2 receptor. Many variations on this type of assay are known in the art and it is common to include a variety of wash steps, to conduct assays with different concentrations of labeled ligand, and to modify incubation conditions for the purpose of optimizing assays. Such optimization is routine in the art and encompassed by the present invention. It is also common to include procedures for determining nonspecific binding. This is typically accomplished by concurrently assaying cells and labeled ligand with a large excess of unlabeled ligand. For example, incubation may be carried out in the presence of a hundredfold excess of unlabeled S protein. The amount of label associated with cells after such an incubation will typically be considered “nonspecific” and subtracted from incubations performed in the absence of an excess of unlabeled ligand for the purpose of determining “specific” binding, i.e., binding occurring at the receptor.

The invention also includes methods of blocking the binding of the SARS virus to host cell receptors by contacting the receptor with an effective amount of an inhibitor of ACE-2. The term “effective amount” refers to sufficient inhibitor to reduce the amount of virus bound by the human ACE-2 receptor by at least 10%, and preferably by 20, 40, 60 or 80%. As will be recognized by one of skill in the art, the virus and inhibitor must be concurrently available to the receptor to allow them to compete for binding in order for this method to work. For example, in vitro, inhibitor and virus might be mixed together in a solution and concurrently exposed to cells known to express receptor. Alternatively, inhibitor may be administered to a subject during the time at which an active SARS infection is taking place. Any inhibitor known in the art for its ability to block the activity of ACE-2 may be used in the present method. One preferred group of inhibitors has been described by Dales, et al. (J. Am. Chem. Soc. 124:11652-11853 (2002)) which is hereby incorporated by reference. These inhibitors of the structure shown below as Formula (I): 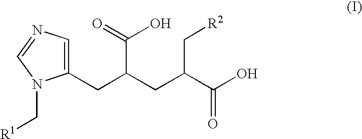
where R1 is a phenyl optionally substituted with one or more groups selected from NO₂Cl, a C₁-C₃ alkyl, or CF₃O—; an alkylcyclohexyl, in which the alkyl is a C₁-C₃ alkyl; or a cyclohexyl optionally substituted with a C₁-C₃ alkyl; and R² is a phenyl or a straight or branched C₁-C₃ alkyl. Other preferred inhibitors include antibodies that bind to ACE-2 and soluble forms of human ACE-2 such as that shown as SEQ ID NO:2.

In addition to including the various methods discussed above, the invention is also directed specifically to a substantially purified protein consisting essentially of the amino acid sequence of SEQ ID NO:2, i.e., the soluble form of human ACE-2. The term “substantially purified” or “substantially pure” as used herein refers to a protein, peptide or polynucleotide that is essentially free from other contaminating components such as proteins, carbohydrates or lipids. One method for determining the purity of a protein or nucleic acid is by electrophoresing a preparation in a matrix such as polyacrylamide or agarose. Purity is evidenced by the appearance of a single dominant band after staining. The term “consisting essentially of” as used herein in connection with human soluble ACE-2 includes polypeptides having the exact sequence shown as SEQ ID NO:2 as well as polypeptides with amino acid differences that are not substantial as evidenced by the polypeptide retaining its basic and novel characteristics. For the purposes of the present invention, the basic and novel characteristic of the soluble ACE-2 protein is its ability to bind with specificity to the SARS S protein. “Specific binding” is defined as occurring in situations where the ACE-2 protein has at least a hundredfold greater affinity for the SARS S protein than for other, unrelated proteins.

The invention includes antibodies made by the process in which an effective amount of the protein of SEQ ID NO:2 is injected into an animal capable of antibody production. Methods for making antibodies are well known in the art and standard methods for making, purifying and testing antibodies may be used.

The invention also includes a substantially purified polynucleotide consisting essentially of nucleotides coding for the protein of SEQ ID NO:2. As used in this connection, the term “consisting essentially of” refers to a sequence of nucleic acids that codes for a soluble human ACE-2 protein having the basic and novel characteristics described above. The invention also includes vectors in which a sequence coding for human soluble ACE-2 is operably linked to a promoter. The vectors may be used for the purpose of transforming host cells capable of making recombinant protein. Among cells preferred for this purpose are human 293 T cells, CHO cells and Vero cells.

The invention also encompasses human soluble ACE-2 protein which has been detectably labeled. This may be added to a test preparation, e.g., a tissue section or sample of biological fluid, to determine whether the SARS virus is present. The presence of the label on cells or virus is indicative of a SARS infection. The most preferred method for carrying out assays of this type involves procedures that have typically been associated with radioimmunoassays or ELISA procedures. In these, a first agent that binds the SARS virus is attached to an immobilized support to form a virus-binding matrix. The support is most typically a multi-well assay plate but other supports such as agarose or acrylamide beads may also be used. The test preparation suspected of containing SARS virus is then incubated with the support matrix under conditions that permit the binding of the virus. Typically, this would involve an incubation in an aqueous buffer at a pH of between 6 and 8 and at a temperature of 15-40° C. After incubation, the immobilized support is separated from the liquid test preparation and, typically, washed one or more times in a buffer. The post-incubation immobilized support then undergoes a second incubation in a liquid containing a second agent that is detectably labeled and which binds either to the first agent or to the SARS virus. After the second incubation, the support is again separated from the incubation fluid and typically undergoes one or more wash steps. An assay is then performed to determine the amount of detectable label that has been bound. In these procedures, either the first agent or the second agent is human ACE-2, preferably the soluble form of human ACE-2 shown as SEQ ID NO:2. Variations on this assay and methods for optimizing results are well known in the art and may be employed in connection with the present invention. For example, it is possible to carry out the assay by first immobilizing the human ACE-2 and examining the ability of the test preparation to displace the binding of labeled SARS S protein or S1 protein. Similarly, the S or S1 protein may be the agent that is immobilized and the ability of test preparations to displace labeled ACE-2 may be measured. In all cases, it is advisable to carry out assays at several different concentrations of test preparation and to include controls for nonspecific binding.

Finally, the present invention includes forms of the ACE-2 protein that, due to deglycosylation at amino acid 90 (Asn, see SEQ ID NO:1), have increased affinity for the SARS S protein. Deglycosylation can either be accomplished enzymatically using enzymes and procedures well known in the art, by recombinantly producing the protein in a cell, e.g., a bacterial cell in which glycosylation does not occur, or by genetically engineering the ACE-2 gene so that a protein is produced that has a residue at position 90 that cannot undergo glycosylation. The deglycosylated protein can be used in assays described above, e.g., assays for identifying inhibitors of interactions between the S protein and the ACE-2 receptor. The deglycosylated protein, or peptides derived therefrom, may also themselves serve to inhibit such interactions.

### DETAILED DESCRIPTION OF THE INVENTION

The present invention is based upon the identification of the biological receptor for the SARS protein. The receptor, ACE-2, is a carboxypeptidase which has previously been described as an essential regulator of heart function. It has been found that transfecting cells with DNA encoding the membrane-bound form of ACE-2 confers upon the cells the ability to become infected with the SARS virus. In addition, it has been found that there is a specific interaction between ACE-2 and the S1 region of the S protein of SARS, as would be expected of the viral receptor. Standard assays for identifying inhibitors of receptor/ligand interaction can be applied to ACE-2 to identify agents of potential therapeutic value. In addition, diagnostic assays utilizing ACE-2 may be applied to the identification of patients infected with the SARS coronavirus and known inhibitors of ACE-2 may be used to prevent viral binding to the S1 protein.

A. Making of Polypeptides and Polynucleotides

The present invention utilizes polynucleotides and polypeptides which encode both ACE-2 and the SARS S protein. The polynucleotides and proteins have been fully described in the art (Marra, et al., Science 300:1399-1404 (2003); Donoghue, et al., Circulation Res. 87(5):E1-E9 (2000)) and may be made using any of the methods that are known in the art with chemical synthesis being generally preferred. The full length sequence of human ACE-2, as well as the SARS S and S1 sequences, are shown herein as SEQ ID NOS:1, 3 and 4, respectively. A fourth protein that has not previously been described in the art, i.e., soluble human ACE-2 is shown as SEQ ID NO:2. Again, these proteins and DNA sequences encoding the proteins may be produced using standard methods. Purification can also be accomplished using standard procedures such as isolating proteins directly from resins used in solid state synthetic methods, using antibodies directed against the polypeptides or by producing the polypeptides in a form in which they are fused to a moiety that aids in purification and which can then be cleaved.

B. Binding Assays

One of the main uses for the ACE-2 receptor and S or S1 ligand is in assays designed to identify agents that block receptor binding. These agents have potential therapeutic application in the treatment of SARS infection. Many different types of binding assays have been described in the art that can be applied to the present invention. For example, cells that either naturally express ACE-2 on their surface or cells that have been engineered to express ACE-2 may be incubated together with detectably labeled S1 protein and with a variety of compounds being tested for binding activity. After incubation, the receptor is separated from the solution containing the other components, e.g., by pelleting cells by centrifugation and then removing the supernatant. The amount of label remaining with the cells can then be determined. The ligand, e.g., S1, may be labeled with the radioisotope of fluorescent label or a chemiluminescent label. Examples of labels that may be used include ¹²⁵I fluorescein, isothiocynate, rhodamine, fluorescamine, luminal and isoluminal. Ligands may also be produced in the form of a fusion protein in which they are joined to a marker amino acid sequence such as that of the Fc IgG fragment. In this case, binding can be quantitated by performing a second incubation in which a labeled antibody is allowed to bind to the marker sequence.

Nonspecific binding in assays may be determined by carrying out the binding reaction in the presence of a large excess of unlabeled ligand. For example, cells expressing ACE-2 may be incubated with labeled SI polypeptide and a hundredfold excess of unlabeled S1 polypeptide. Nonspecific binding may be subtracted from total binding, i.e., binding in the absence of unlabeled peptide, to arrive at specific binding for each sample tested. Other steps, such as washing, stirring, shaking, filtering and the like, may be included in the assays as necessary. Typically, wash steps are included after the separation of membrane-bound ligand from ligand remaining in solution and prior to quantitation. The specific binding obtained in the presence of the test compound is compared with that obtained in the presence of labeled ligand alone to determine the extent to which the test compound has interacted with the receptor.

The assay described above can be varied in many different ways that are known in the art. For example, fluorescent activated cell sorting assays can be adapted for determining receptor binding and it is possible to label test compounds rather than ligand. It will also be recognized by those of skill in the art that any ligand derived from the S protein which maintains its ability to bind to ACE-2 could be labeled and used as the ligand in assays. Preferably, test compounds are examined at several different concentrations and those that show inhibitory activity are further examined for binding affinity and specificity. Inhibitory peptides can also be examined using intact virus. Those showing strong activity and a high degree of specificity have potential as therapeutic agents for treating viral infection.

C. Inhibitors of the Binding of S to ACE-2

Inhibitors of ACE-2 have been described by Huang, et al. (J. Biol. Chem. 278(18):15532-15540 (2003)) and by Dales, et al. (J. Am. Chem. Soc. 124:11852-11853 (2002)). These inhibitors may be made and used in accordance with the guidance provided by the relevant references, both of which are incorporated herein by reference.

Inhibitors may also take the form of antibodies that bind specifically to ACE-2. Specific binding in this sense refers to antibodies that have at least a hundredfold greater affinity for ACE-2 than to other structurally unrelated proteins. The antibodies may be produced by injecting the ACE-2 protein itself into an appropriate animal or, alternatively, injecting short peptides made to correspond to different regions of ACE-2. The peptides should be at least five amino acids in length and should be selected from regions believed to be unique to the ACE-2 protein. Thus, highly conserved regions, e.g., transmembrane regions, should generally be avoided in selecting peptides for the generation of antibodies. Methods for making and-detecting antibodies are well known to those of skill in the art and as evidenced by standard reference works such as: Harlow, et al., Antibodies, A Laboratory Manual, Cold Spring Harbor Laboratory, N.Y. (1988); and Campbell, “Monoclonal Antibody Technology,” in Laboratory Techniques in Biochemistry and Molecular Biology (1984).

“Antibody” as used herein is meant to include intact molecules as well as fragments which maintain their ability to bind to antigen, e.g., Fab and F(ab)₂ fragments. These fragments are typically produced by proteolytically cleaving intact antibodies using enzymes such as papain (to produce Fab fragments) or pepsin (to produce F(ab)₂ fragments). The term “antibody” also refers to both monoclonal antibodies and polyclonal antibodies. Polyclonal antibodies are derived from the sera of animals immunized with the antigen. Monoclonal antibodies can be prepared using hybridoma technology (Kohler, et al., Nature 256:495 (1975); Hammerling, et al., in Monoclonal Antibodies and T-Cell Hybridomas, Elsevier, N.Y., pp. 563-681 (1981)). The ability of the antibodies to block the binding of the SARS S or S1 protein to cellular receptors can be tested using the binding assays described above. Antibodies may also be incubated with the SARS virus to directly determine their ability to prevent the virus from binding to cells susceptible to infection.

D. Use of Labeled Soluble ACE-2

As described above, the soluble form of ACE-2 (SEQ ID NO:2) may be used to block the binding of S or S1 to cellular receptors. The soluble protein can also be detectably labeled and used in assays to determine whether SARS virus is present, e.g., in a biological sample. This could be done using a fluorescence activated cell sorting assay, or histologically. Preferably, assays are carried out in essentially the same format as radioimmunoassays or immunometric assays, but using labeled soluble ACE-2 in the place of labeled antibody. Typically, these assays involve attaching an agent that binds to the SARS virus to an immobilized support, e.g., a multiwell assay plate. An incubation is then performed using a liquid preparation which is suspected of containing SARS virus. After the incubation, the immobilized support is separated from the incubation solution and, after washing, is incubated with a solution containing the detectably labeled soluble ACE-2. After the second incubation, unbound material is removed and the amount of label associated with the immobilized support is determined.

Many variations on this type of assay are possible and would be readily apparent to one of skill in the art. For example, unlabeled ACE-2 can be attached to the immobilized support and the presence of virus determined using a detectably labeled antibody. Similarly, virus or cells suspected of containing virus can be directly immobilized and then examined using labeled peptide or labeled antibody.

### EXAMPLES

The present example describes the identification of angiotensin-converting enzyme-2 (ACE-2) isolated from SARS coronavirus-permissive Vero E6 cells as efficiently binding the S1 domain of the SARS protein. A soluble form of ACE-2, but not that of a related enzyme, ACE-1, blocked association of the S1 domain with Vero E6 cells. 293 T cells transfected with ACE-2, but not cells transfected with HIV-1 receptors, formed syncytia with S-protein-expressing cells. Finally, SARS coronavirus replicated efficiently on ACE-2 transfected 293 T cells, but not cells that were mock-transfected. Based upon these results it was concluded that ACE-2 is a functional receptor for the SARS coronavirus.

A. Methods

Immunoprecipitation and Identification of ACE-2 from Vero E6 Cells

Vero E6 cells were metabolically labeled for 48 hours with [³⁵S-cysteine and -methionine and lysed in 1.5 ml per 100-mm dish of 0.3% n-decyl-β-D-maltopyranoside (DDM, Anatrace) in phosphate buffered saline (PBS) containing protease-inhibitor cocktails (Sigma and Roche) and 2 mM phenylmethylsulfonylfluoride (PMSF, Sigma). Following removal of cell debris by centrifugation, lysate was incubated for 1 hour at room temperature with 2.5 μg of purified S1-Ig or IFNAR2-Ig fusion constructs and Protein-A Sepharose. Alternatively, C-terminally C9-tagged forms of the S1 domain (S1-C9) or of controlled protein (HIV-1 gp120-C9, IFNAR2-C9) were incubated with the antibody 1D4 (National Cell Culture Center) together with Protein-A Sepharose. Precipitates were washed twice in 0.3% DDM/PBS and once in PBS alone. Bound proteins were eluted in reducing Lammlie sample buffer at 55° C. or in non-reducing buffer at 37° C., for 10 minutes. Proteins were separated by SVS-PAGE on an 8% Tris-Glycine gel (Invitrogen). Using this approach, approximately 5×10⁷ unlabeled Vero E6 cells were used to generate a distinct band of 110 kD that could be readily visualized by Coomassie staining. This band was excised from the gel and incubated with trypsin and masses of tryptic fragments were determined by Matrix-assisted Laser Desorption Ionization Time-of-Flight Mass Spectrometry (MALDI-TOF MS). The masses were compared with possible tryptic fragments of proteins in-the GenBank database using Sequest software.

Measurement of Receptor Expression on Vero E6 and 293 T Cells

Plasmids encoding the signal sequence of CD5 and a fusion of the S1-domain of the SARS coronavirus (SARS-CoV) protein (residues 12-672), or the first 316 residues of that domain (12-327), with the Fc region of human IgG1 (S1-Ig and S1(327)-Ig, respectively), transfected into 293 T cells and Ig-fusion proteins were purified on Protein-A Sepharose beads. 5×10⁵ 293 T cells transfected with ACE-2-expressing or controlled plasmids, or the same number of untransfected Vero E6 cells, were incubated with 15 μg per ml of S1-Ig or S1(327)-Ig in a volume of 100 μl. In some cases, 15 μg per ml of soluble forms of ACE-1 or ACE-2 (R&D Systems) was also included. Cells were washed in PBS with 0.5% BSA and 0.1% NaN₃, incubated with FITC labeled goat anti-human IgG and analyzed by FACS. Plasmid-expressing soluble or full-length ACE-2 was PCR amplified from human lung cDNA (Clontech) and ligated into the pc DNA3.1 vector.

Syncytia Formation Between SARS-CoV S-Protein- and ACE-2-Expressing Cells

293 T cells, approximately 50% confluent on a 6-well plate were transfected by the calcium-phosphate method with plasmids encoding a codon-optimized form of the SARS-CoV S-Protein, or of the HIV-1 envelope glycoprotein gp160 (ADA, Isolate). In parallel, 293 T or HeLa cells were transfected with plasmids encoding ACE-2 or the HIV-1 receptors CD4 and CCR5. One day after transfection, cells were trypsinized and those expressing viral-envelope proteins were mixed with cells expressing receptors at a 1:1 ratio, and plated on 12- or 24-well plates. 24-48 hours after mixing, multinucleated giant cells were observed and counted. In some cases, soluble forms of ACE-1 or ACE-2 (R&D Systems) were added at the time of cell mixing.

Infection of Mock- and ACE-2 Transfected 293 T Cells

SARS-CoV (Urbani strain) was passaged on Vero E6 cells. 293 T cells transfected in 25-cm² flasks with pcDNA3.1 alone or with pcDNA3.1 expressing ACE-2 were infected with 1.4×10⁶ TCID₅₀ of SARS-CoV, as measured by endpoint titration on Vero E6 cells, or left uninfected, and washed twice in culture medium. Cells were monitored for CPE for 4 days. Aliquots of cell supernatants were harvested 0, 2, and 4 days following infection and wash. RNA was recovered using a viral RNA mini-prep kit (Quiagen). Semi-quantitative RT-PCR was performed using a nested protocol as described by Drosten, et al. (N. Engl. J. Med. 348:1967-1976 (2003)). Virus titration was performed by feeding 5×10³ Vero E6 cells per well in 96-well microtiter plates one day prior to infection. Culture supernatant from infected 293 T cells was added to the first wells in triplicate and serially diluted. The assay was read for viral CPE at 3 days and infectious titer calculated using the Spearman-Karber method.

B. Results

The African Green Monkey kidney cell line Vero E6 has been demonstrated to permit replication of SARS-CoV (Ksiazek, et al., N. Engl. J Med. 348:1953-1966 (2003)). In the present experiments, it was first determined that a protein expressing residues 12-672 of the SARS-CoV S protein fused to the Fc domain of human IgG1 (S1-Ig) specifically recognized a moiety present on Vero E6 cells but did not bind to human 293 T cells. Using this same fusion protein or a C-terminally tagged form of the S1 domain, a protein band of approximately 110 kD could be immunoprecipitated from metabolically labeled Vero E6 cells lysed with 0.3% n-decyl-β-D-multopyranoside in phosphate-buffered saline. When the immunoprecipitated protein was incubated with PNGase F, an enzyme that removes N-glycosylation, two bands were observed at approximately 80-85 and 100 kD.

Analysis of the 110 kD band was carried out by trypsin digestion and mass spectrometry. Three human proteins were identified whose sequences are consistent with the masses of tryptic fragments obtained from this band. Two of these, myosin 1b and major vault protein, do not localize to the cell surface and are ubiquitously expressed, and therefore were not further analyzed. Ten trypsin fragments consistent with sequences comprising 17% of the amino acid sequence of ACE-2 were also identified. Because the tissue distribution and subcellular localization of ACE-2 were appropriate for a receptor of SARS-CoV, it was cloned from cDNA obtained from human lung for further analysis.

It was found that 293 T cells transfected with plasmid expressing ACE-2, but not those transfected with vector alone, were specifically recognized by S1-Ig. A soluble form of ACE-2, but not that of a related enzyme, ACE-1, blocked association of S1-Ig with Vero E6 cells. Finally, S1-Ig could immunoprecipitate a soluble form of ACE-2 also recognized by an anti-ACE-2 antibody. When the approximately 110 kD soluble form of ACE-2 was incubated with PNGase F, an 85 kD band was observed, suggesting that the lower band observed in PNGase F-treated Vero E6 immunoprecipitates is ACE-2. These data demonstrate a specific, high-affinity association between the S1 domain of the SARS-CoV protein and ACE-2, but not ACE-1.

Proteins that mediate fusion between viral and cellular membranes can, in some cases, also do so between cells that express the viral fusion protein and those that express the viral receptor. For instance, cells expressing HIV-1 envelope glycoprotein gp160 can form multinucleated syncytia with cells expressing the HIV-1 receptors CD4 and CD5. Because syncytia have been observed in Vero E6 cultures infected with SARS-CoV in SARS-CoV infected primates and in SARS patients (Ksiavek, et al., N. Engl. J. Med. 348:1953-1966 (2003); Kuiken, et al., Lancet 362:263-270 (2003)), we investigated whether SARS-CoV S protein-expressing 293 T cells could fuse with 293 T cells expressing ACE-2. It was found that these cells formed syncytia efficiently. As expected, 293 T cells transfected with CD4 and CCR5 formed many syncytia with cells expressing HIV-1 gp160, but not with SARS-CoV S protein-expressing cells. In contrast, 293 T cells expressing ACE-2 did not form syncytia with cells expressing gp160, but formed many large syncytia with cells expressing S protein. 293 T cells expressing S protein also efficiently formed syncytia with ACE-2- but not mock-transfected HeLa cells. Syncytia formation mediated by the SARS-CoV S protein was partially blocked by soluble ACE-2 and by S1-Ig, but not by soluble ACE-1 or soluble CV4. These data demonstrate that the ability of the SARS-CoV S protein to mediate cell-cell fusion is dependent on the presence of ACE-2.

The ability of ACE-2 to mediate viral replication was also investigated. ACE-2- and mock-transfected 293 T cells were incubated in the presence or absence of SARS-CoV. After 2 days in culture, numerous detached, round, and floating cells were observed in cultures of infected ACE-2 transfected cells, consistent with viral replication. In contrast, infected mock-transfected cells and uninfected ACE-2-transfected cells were indistinguishable from uninfected control cells, with none of the signs of cytopathicity observed in infected ACE-2 expressing cells. ACE-2- but not mock-transfected 293 T cells supported efficient replication of SARS-CoV. Viral genomic RNA in cell supernatants was measured by semi-quantitative RT-PCR. Viral genome copies in ACE-2-transfected cells increased more than 10,000-fold between day 0 and day 2. In contrast, genome copies in mock-transfected cells increased ten-fold in the same period, consistent with some basal replication on 293 T cells. No copies were observed in supernatants from uninfected cells. In addition, virus in supernatants from infected ACE-2- and mock-transfected cells was measured by titration onto Vero E6 cells. Virus stock obtained at day 4 from ACE-2 transfected cells was found to efficiently replicate on Vero E6 cells, with visible cytopathic effect (CPE) up through and including the highest dilution assayed (1:6561), whereas no CPE was observed from virus stocks obtained from mock-transfected cells beyond a 1:27 dilution. These data show that ACE-2 makes a critical contribution to SARS-CoV replication.

C. Discussion

The experiments described above show that ACE-2 can be immunoprecipitated from Vero E6 cells by the S1 domain of the SARS-CoV S protein, that it mediates fusion with S-protein-expressing cells, and that it promotes viral replication in a cell line otherwise inefficient for SARS-CoV infection. It may therefore be concluded that ACE-2 is a functional receptor for SARS-CoV. Real-time PCR analysis of 72 human tissues has demonstrated efficient expression of ACE-2 mRNA in the bronchus and lung parenchyma as well as in the heart, kidney, and gastrointestinal tract (Harmer, et al., FEBS Lett. 532:107-110 (2002)). Lung and kidney are also the primary sites of expression of murine ACE-2 (Komatsu, et al., DNA Seq. 13:217-220 (2002)). This tissue distribution is consistent with the pathology of SARS, which is characterized by an acute, uncontrolled infection of the lungs. SARS-CoV has also been found in kidney tissue of infected patients and replicates efficiently on primate kidney-cell lines FRhK4 and Vero E6. Detection of virus in the feces may reflect expression of ACE-2 message in the colon and rectum (Drosten, et al., N. Engl. J. Med. 348:1967-1976 (2003); Harmer, et al., FEBS Lett. 532:107-110 (2002)).

All references cited herein are fully incorporated by reference. Having now fully described the invention, it will be understood by those of skill in the art that the invention may be performed within a wide and equivalent range of conditions, parameters and the like, without affecting the spirit or scope of the invention or any embodiment thereof.

1. A method of growing the SARS coronavirus in cells, comprising
a) engineering said cells to stably express the human ACE-2 protein;
b) culturing said cells under conditions that promote cellular growth;
c) infecting said cells with said SARS coronavirus; and
d) harvesting said virus from said cells.

2. The method of claim 1, wherein said cells are engineered by transfecting them with a vector comprising a promoter operably linked to a sequence coding for the human ACE-2 protein.

3. The method of claim 1, wherein said cells are mammalian cells.

4. The method of claim 1, wherein said cells are selected from the group consisting of:
human 293 T cells; CHO cells; and Vero cells.

5. A method of assaying a test compound for its ability to block the binding of the SARS protein to the human ACE-2 receptor, comprising:
a) incubating cells expressing human ACE-2 on their surface with:
i) a detectably labeled ligand selected from the group consisting of: the SARS S protein; and a detectably labeled fragment of the SARS S protein that maintains the ability to bind with specificity to human ACE-2;
ii) said test compound;
b) determining the amount of detectably labeled ligand bound to said cells;
c) comparing the results obtained in step b) with those obtained from cells incubated under similar conditions but in the absence of said test compound; and
d) concluding that said test compound blocks the binding of the SARS S protein to human ACE-2 if the amount of binding observed in the presence of said test compound is lower than in the absence of said test compound.

6. The assay of claim 5, wherein said cells expressing human ACE-2 on their surface are cells that have been transfected with a vector comprising a promoter operably linked to a sequence coding for human ACE-2.

7. The assay of either claim 5 or claim 6, wherein said cells are incubated with detectably labeled SARS S protein.

8. The assay of either claim 5 or claim 6, wherein said cells are incubated with detectably labeled SARS S1 protein;

9. A method for blocking the binding of the SARS virus to a host cell, comprising contacting said host cell with an effective amount of an inhibitor of ACE-2.

10. The method of claim 9, wherein said inhibitor of ACE-2 has the structure of Formula (I): 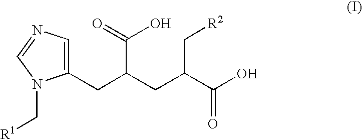
wherein:
R¹ is selected from the group consisting of: phenyl optionally substituted with a group selected from NO₂Cl, a C₁-C₃ alkyl, and CF₃O—; an alkylcyclohexyl, wherein said alkyl is a C₁-C₃ alkyl; and cyclohexyl optionally substituted with a C₁-C₃ alkyl; and
R² is selected from the group consisting of phenyl and a straight or branched C₁-C₃ alkyl.

11. The method of claim 9, wherein said inhibitor of ACE-2 is an antibody.

12. The method of claim 9, wherein said inhibitor of ACE-2 is soluble ACE-2 as shown in SEQ ID NO:2.

13. A substantially purified protein, consisting essentially of the amino acid sequence of SEQ ID NO:2.

14. An antibody made by the process of administering an effective amount of the protein of claim 13 to an animal capable of antibody production.

15. A substantially purified polynucleotide consisting essentially of nucleotides encoding the protein of SEQ ID NO:2.

16. A vector comprising a promoter operably linked to a coding sequence, wherein said coding sequence encodes a protein having the sequence of SEQ ID NO:2.

17. A host cell transformed with the vector of claim 16.

18. Soluble ACE-2 consisting essentially of the amino acid sequence of SEQ ID NO:2, wherein said soluble ACE-2 is detectably labeled.

19. A method for detecting the presence of SARS coronavirus in a test preparation, comprising:
a) adding the detectably labeled soluble ACE-2 of claim 18 to said test preparation; and
b) determining the amount of labeled soluble ACE-2 bound by cells or virus in said preparation.

20. The method of claim 19, wherein said test preparation is a biological fluid or tissue sample.

21. A method of detecting the presence of SARS coronavirus in a liquid test preparation, comprising:
a) attaching a first agent to an immobilized support, wherein said first agent binds to SARS coronavirus to form a virus-binding immobilized support;
b) incubating said liquid test preparation with said virus-binding immobilized support under conditions promoting the binding of virus;
c) after the incubation of step b), removing liquid test preparation to leave behind a post-incubation immobilized support;
d) incubating said post-incubation immobilized support with a post-incubation liquid preparation comprising a second agent, wherein said second agent is detectably labeled and binds to either said first agent or to SARS virus.
e) removing said post-incubation liquid preparation from said post-incubation immobilized support to form a final immobilized support;
f) assaying said final immobilized support to determine the amount of detectably labeled second agent present;
and wherein either said first agent or said second agent is soluble or insoluble ACE-2.

22. The method of claim 21, wherein either said first agent or said second agent is soluble ACE-2 as shown in SEQ ID NO:2.
